(19) United States
(12) Patent Application Publication
(10) Pub. No.: US 2019/0224117 A1
(43) Pub. Date: Jul. 25, 2019

(54) Cannabis Extract Chewing Gum

(71) Applicants: Yury Leon Shmerlis, Tampa, FL (US); Anna Proshak, New York, NY (US)

(72) Inventors: Yury Leon Shmerlis, Tampa, FL (US); Anna Proshak, New York, NY (US)

(21) Appl. No.: 16/369,875

(22) Filed: Mar. 29, 2019

Related U.S. Application Data
(63) Continuation-in-part of application No. 15/622,576, filed on Jun. 14, 2017.
Application No. 15/622,576 is a continuation-in-part of application No. 15/622,587, filed on Jun. 14, 2017, now abandoned.

(51) Int. Cl. A61K 9/68; A23G 4/06; A23G 4/10; A23G 4/08; A61K 36/185; A61K 31/352; A61K 31/05; A61K 31/522; A61K 31/12

(52) U.S. Cl. CPC A61K 9/0058; A23G 4/068; A23G 4/10; A23G 4/08; A23V 2002/00; A61K 31/352; A61K 31/05; A61K 31/522; A61K 31/12; A61K 36/185

(57) ABSTRACT

A cannabis extract chewing gum for delivering cannabis extracts, such as tetrahydrocannabinol (THC) oil or cannabidiol (CBD) oil, to a user. The cannabis extract chewing gum may comprise a gum base, cannabis extract, flavorings, sweeteners, fixatives, and other additives. The cannabis extract chewing gum provides an effective and healthful way for users to easily absorb THC or CBD without the harmful effects of other common means of administering cannabinoids, such as smoking or vaping.

### CROSS-REFERENCE TO RELATED APPLICATIONS

This Application is a Continuation-in-Part of both U.S. Non-Provisional patent application Ser. No. 15/622,576, filed on Jun. 14, 2017, entitled “Cannabidiol Chewing Gum”, by inventor Yury Leon Shmerlis, and U.S. Non-Provisional patent application Ser. No. 15/622,587, filed on Jun. 14, 2017, entitled “Tetrahydrocannabinol Chewing Gum”, by inventor Yury Leon Shmerlis, the contents of both of which are expressly incorporated herein by this reference as though set forth in their entirety, and to which priority is claimed.

### FIELD OF USE

The present disclosure relates generally to chewing gum comprising one or more cannabis extracts. More particularly, the present disclosure relates to chewing gum compositions comprising cannabidiol (CBD) or tetrahydrocannabinol (THC) for orally delivering CBD and/or THC to a user.

### BACKGROUND

Cannabis, which contains many chemical compounds useful for medicinal or recreational application, may be treated in a large variety of methods to create cannabis extracts. Cannabis extracts are useful for a variety of medicinal or recreational applications, due to their high concentration of cannabinoids (chemical compounds which alter neurotransmitter release in the brain) as a result of the extraction process.

Numerous methods exist for creating cannabis extracts from raw cannabis plant matter. For example, volatile solvents such as butane, propane, and hexane are often used to prepare cannabis extract, though supercritical fluid extraction using carbon dioxide may be used to alleviate concerns of fire and explosion, while still resulting in a high-quality product.

The resulting cannabis extract (also referred to as marijuana concentrate, extract, shatter, wax, butane hash oil, butane honey oil, live resin, budder, taffy, etc.) may take a variety of forms, such as solids, powders, or waxes. These cannabis extracts are highly potent, due to their high concentration of cannabinoids, a diverse group of naturally-occurring chemicals in the cannabis plant which alter neurotransmitter release in the brain. Of the at least 113 cannabinoids present in cannabis, tetrahydrocannabinol (THC) and cannabidiol (CBD) are two of the most frequently used and sought after.

Regardless of the exact method used to create a cannabis extract, the result is highly potent: cannabis extracts may contain extraordinarily high THC levels that are capable of ranging from 40% to 80%, up to four times stronger in THC content than high grade/premium marijuana, which normally measures around 20% THC levels. Some cannabis extracts may now have a 99.58% THC content.

Tetrahydrocannabinol (THC) is the main psychoactive component of cannabis. A psychoactive component is a chemical substance that alters brain function and induces changes in perception or mood of a user. Although cannabis is primarily used as a recreational drug, the component THC has several medical applications. Traditionally, THC and its isomers have been known to treat anorexia in people with Acquired Immune Deficiency Syndrome (AIDS). Additionally, THC has been used to treat refractory nausea and vomiting in patients undergoing chemotherapy. THC has also been used to alleviate pain in patients who suffer from neuropathic pain, spasticity, overactive bladder, etc.

Although cannabis is frequently used as a recreational drug (via THC, as discussed above), cannabis has applications related to CBD, as well. The cannabinoid CBD does not cause the “high” associated with the tetrahydrocannabinol (THC) component of cannabis. CBD has been considered to have a wide scope of applications to multiple medical conditions. Compared to THC, CBD has increased potential scope for applicability, as CBD has little or no side effects and does not interfere with the psychomotor learning and psychological functions of users. Furthermore, CBD has been used to treat certain forms of epilepsy. Typically, CBD constitutes up to forty percent of the extract from the cannabis plant.

Existing methods of absorbing THC or CBD require a user to smoke, vaporize, or ingest cannabis plant matter. Using this method affects the lungs of the user, especially if the user has a related breathing condition. Moreover, smoking may have undesirable harmful effects on the user, such as contracting a severe cough. For patients already suffering from pain due to their medical condition, this is highly disadvantageous.

Hence, there is a long felt but unresolved need for a composition and method of ingesting THC and/or CBD which eliminates the need to smoke, vaporize, or eat cannabis plant matter.

### SUMMARY OF EMBODIMENTS

The following presents a simplified overview of the example embodiments in order to provide a basic understanding of some embodiments of the example embodiments. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented hereinbelow. It is to be understood that both the following general description and the following detailed description are exemplary and explanatory only and are not restrictive.

To minimize the limitations in the prior art, and to minimize other limitations that will become apparent upon reading and understanding the embodiments, the present specification discloses a new and useful cannabis extract chewing gum, comprising cannabis extract, gum base, fixing agent, gingerol, caffeine, and various flavorings and sweeteners. The cannabis extract may contain tetrahydrocannabinol (THC) or cannabidiol (CBD). The user ingesting the cannabis extract chewing gum may receive THC and/or CBD without the need to smoke, vape, vaporize, atomize, or ingest cannabis plant matter.

The embodiments disclosed herein are preferably designed to offer an innovative, applicable, and practical way for chewing gum users to ingest THC and/or CBD without the need to smoke, vaporize, or ingest cannabis plant matter.

It is an object to overcome the limitations of the prior art.

One embodiment of the present disclosure may be a composition comprising: a cannabis extract chewing gum containing CBD. The cannabis extract chewing gum preferably comprises mint and/or sweetener which meld in such a way that is smooth to the taste, and that the chewing gum does not have a bitter or off taste. The cannabis extract chewing gum may comprise: a gum base, CBD oil, flavoring, sweetener, gingerol, and a fixing agent. The gum base may be approximately 25.0-90.0 wt %, the CBD oil may be approximately 5.0-15.0% wt %, the flavoring may be approximately 5.0-15.0 wt %, the sweetener may be approximately 0.001-50.0 wt %, the gingerol may be approximately 1.0-20.0 wt % and the fixing agent may be approximately 5.0-15.0 wt %. In some embodiments, the fixing agent may be a polyol, such as niaouli alcohol, isopulegol, sorbitol, maltitol/isomalt, mannitol, or starch. The fixing agent may allow the gum to have a hard outer shell. The gum may also comprise other additives, softeners, plasticizers, glycerine, flavorings, coloring agents, and sweeteners.

One embodiment of the present disclosure may be a composition comprising: a cannabis extract chewing gum containing THC. The cannabis extract chewing gum preferably comprises mint and/or a sweetener which meld in such a way that is smooth to the taste, and that the chewing gum does not have a bitter or off taste. The cannabis extract chewing gum may comprise: a gum base, THC oil, flavoring, sweetener, caffeine, and a fixing agent. The gum base may be approximately 25.0-90.0 wt %, the THC oil may be approximately 5.0-15.0% wt %, the flavoring may be approximately 5.0-15.0 wt %, the sweetener may be approximately 0.001-50.0 wt %, the caffeine may be approximately 1.0-20 wt %, and the fixing agent may be approximately 5.0-15.0 wt %. The gum may also comprise other additives, softeners, plasticizers, glycerine, flavorings, coloring agents, and sweeteners.

Another embodiment may be a process for making a cannabis extract chewing gum, the steps comprising: providing a gum base, CBD oil, mint oil, stevia, gingerol, and a fixing agent; adding the gum base to a mixer, and mixing the gum base for a duration sufficient to convert the gum base from individual pellets or balls of gum base into a semi-consistent, rubbery solid; adding, while the gum base continues to be mixed, any desired flavors or other additives (such as CBD oil, mint oil, caffeine, gingerol, and stevia), to the mixer; stirring the gum base, flavors and additives together until a homogeneous rubbery solid is obtained; transferring the homogeneous rubbery solid to a conveyer belt; running the homogeneous rubbery solid through a conveyer belt such that the homogeneous rubbery solid mixture is compressed into a relatively thin gum product sheet; and processing the thin gum product sheet by mechanically separating the thin gum product sheet into sufficiently small pieces fit for easy human chewing and use. The fixing agent may be used to create a hard outer coating on the gum.

Another embodiment may be a process for making a cannabis extract chewing gum, the steps comprising: providing a gum base, THC oil, mint oil, stevia, and a fixing agent; adding the gum base to a mixer, and mixing the gum base for a duration sufficient to convert the gum base from individual pellets or balls of gum base into a semi-consistent, rubbery solid; adding, while the gum base continues to be mixed, any desired flavors or other additives (such as THC oil, mint oil, caffeine, gingerol, and stevia), to the mixer; stirring the gum base, flavors and additives together until a homogeneous rubbery solid is obtained; transferring the homogeneous rubbery solid to a conveyer belt; running the homogeneous rubbery solid through a conveyer belt such that the homogeneous rubbery solid mixture is compressed into a relatively thin gum product sheet; and processing the thin gum product sheet by mechanically separating the thin gum product sheet into sufficiently small pieces fit for easy human chewing and use.

These, as well as other components, steps, features, objects, benefits, and advantages, will now become clear from a review of the following detailed description of illustrative embodiments and the claims.

### DETAILED DESCRIPTION OF ILLUSTRATIVE EMBODIMENTS

In the following detailed description of various embodiments, numerous specific details are set forth in order to provide a thorough understanding of various aspects of the embodiments. However, the embodiments may be practiced without some or all of these specific details. In other instances, well-known procedures and/or components have not been described in detail so as not to unnecessarily obscure aspects of the embodiments.

While some embodiments are disclosed here, other embodiments will become obvious to those skilled in the art as a result of the following detailed description. These embodiments are capable of modifications of various obvious aspects, all without departing from the spirit and scope of protection. Also, the reference or non-reference to a particular embodiment shall not be interpreted to limit the scope of protection.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the terms “approximately” and “about” generally refer to a deviance of within 5% of the indicated number or range of numbers. In one embodiment, the term “approximately” and “about”, may refer to a deviance of between 0.0001-10% from the indicated number or range of numbers.

As used herein, the term “gum base” generally refers to a chewable rubber or plastic, which provides the structural integrity of the cannabis extract chewing gum. It may comprise either natural materials (such as the plant resin chicle) or synthetic materials (such as, for example, paraffin wax and related polymers). The gum base may further comprise a combination of elastomers, resins, waxes, fats, emulsifiers, fillers, or antioxidants as desirable to modify the texture of the cannabis extract chewing gum.

As used herein, the term “cannabis extract” refers to a concentrated, purified form of either THC or CBD, which has been extracted from the cannabis plant. It is contemplated that, in some embodiments, the cannabis extract may take the form of a concentrated oil, which may be readily poured or added dropwise to the other gum ingredients during the mixing process. The purity of the cannabis extract may range from 50-99%, and is typically 90% pure. Typically, the cannabis extract is CBD oil or THC oil.

As used herein, the term “fixing agent” generally refers to a polyol that allows the gum to have a hard outer coating. The polyol may be Sorbitol, Maltitol/Isomalt, Mannitol, Starch, and the like.

As used herein, the terms “sweeteners” and “flavors” generally refer to compounds added in order to improve the taste of the cannabis extract chewing gum. It is contemplated that various common sweeteners, such as xylitol and stevia, may be used to enhance the taste of the cannabis extract chewing gum. Other sweeteners used may include: sugar, dextrose, glucose or corn syrup, erythritol, isomalt, maltitol, mannitol, sorbitol, lactitol, aspartame, acesulfame-K, saccharine, sucralose, neohesperidine, dihydrichalcone. Other flavors used may be natural or synthetic and may be peppermint, spearmint, or a sour acid, such as citric, tartaric, malic, lactic, adipic, and fumaric.

As used herein, the term “additives” generally refers to non-structural or flavor-related compounds that are added to the cannabis extract chewing gum to impart other desirable properties. These may include various vitamins for imparting healthful effects to the user. Caffeine and gingerol are two additives that might be used.

The gum of the present disclosure may also have a softener or plasticizer, such as lecithin, hydrogenated vegetable oils, glycerol ester, lanolin, methyl ester, pentaerythritol ester, rice bran wax, stearic acid, sodium potassium stearates, and the like.

The present disclosure relates to a chewing gum that contains a cannabis extract that delivers CBD and/or THC to the user.

The cannabis extract chewing gum may further comprise gingerol, caffeine, flavorings, and sweeteners, preferably artificial sweeteners. The user masticating the cannabis extract chewing gum receives THC and/or CBD from the chewing gum. The artificial sweeteners may be based on a preference of the user. Moreover, the chewing gum may also comprise vitamins, antibiotics, colorants, etc. The cannabis extract chewing gum ensures delivery of THC/CBD to the user who chews it. In some embodiments, the addition of vitamins or antibiotics may provide added benefits to users. When a user chews the cannabis extract chewing gum with THC and/or CBD, the absorption may happen through the lining of the mouth, in addition to what might be swallowed with saliva.

One embodiment of the present disclosure is a cannabis extract chewing gum, comprising a gum base, CBD oil, mint oil, stevia, an additive, such as gingerol, and a fixing agent.

Table 1 shows the preferred ingredients of one embodiment of the CBD cannabis extract chewing gum product.

TABLE 1
CBD Cannabis Extract Chewing Gum Recipe

| | | % wt/wt | |
|---|---|---|---|
| | % wt/wt | range (wt | |
| Ingredient | (wt %) | % range) | Purpose |
| Gum Base | 65 | 25.0-90.0 | Provides textural support for |
| | | | chewing and carrying other |
| | | | ingredients |
| CBD Oil | 10 | 5.0-15.0 | Health benefits |
| Mint oil | 10 | 5.0-15.0 | Flavor |
| Additive | 5 | 1.0-20.0 | Additional health benefits. |
| (typically | | | |
| gingerol is | | | |
| combined with | | | |
| CBD, but | | | |
| caffeine may | | | |
| also be used) | | | |
| Stevia | 5-10 | 0.0001-50.0 | Sweetener |
| Fixing Agent | 5 | 0-15.0 | Provide hard outer shell. |

Another embodiment of the present disclosure is a cannabis extract chewing gum, comprising a gum base, THC oil, mint oil, stevia, an additive, such as caffeine and a fixing agent.

In one embodiment, the cannabis extract chewing gum may be produced by the following steps:
Providing a gum base, CBD oil, mint oil, stevia, and a fixing agent;
Adding the gum base to a mixer;

Mixing the gum base for a duration sufficient to convert the gum base from individual pellets or balls into a semi-consistent, rubbery solid;
Adding CBD oil, mint oil, stevia, caffeine and/or gingerol, and fixing agent to the mixture
Stirring the CBD oil, mint oil, stevia, caffeine and/or gingerol, fixing agent, and gum base together until a homogeneous rubbery solid develops;
Transferring said homogeneous rubbery solid to a conveyer belt;
Feeing said homogeneous rubbery solid through a compressing bottleneck in said conveyer belt, such that the resulting gum mixture is pressed into thin sheets; and
Mechanically separating the thin gum sheets into small pieces fit for easy human chewing and use.

In the above embodiment, the gum base may be mixed for a duration ranging from 15-90 minutes with a temperature range of 75-180 C. The above embodiment is preferably made with ingredient proportions as laid out in Table 1, above.

Table 2 shows the preferred ingredients of one embodiment of the THC gum product.

TABLE 2
THC Gum Recipe

| | | % wt/wt | |
|---|---|---|---|
| | % wt/wt | range (wt | |
| Ingredient | (wt %) | % range) | Purpose |
| Gum Base | 65 | 25.0-90.0 | Provides textural support for |
| | | | chewing and carrying other |
| | | | ingredients |
| THC Oil | 10 | 5.0-15.0 | Health benefits |
| Mint oil | 10 | 5.0-15.0 | Flavor |
| Additive | 5 | 1.0-20.0 | Additional health benefits. |
| (typically | | | |
| caffeine is | | | |
| combined with | | | |
| THC, but | | | |
| gingerol may | | | |
| also be used) | | | |
| Stevia | 5-10 | 0.0001-50.0 | Sweetener |
| Fixing Agent | 5 | 0-15.0 | Provide hard outer shell |

Another embodiment of the present disclosure is a cannabis extract chewing gum, comprising a gum base, THC oil, mint oil, stevia, an additive, such as caffeine, and a fixing agent.

In one embodiment, the cannabis extract chewing gum may be produced by the following steps:
Providing a gum base, CBD oil, mint oil, stevia, and a fixing agent;
Adding the gum base to a mixer;
Mixing the gum base for a duration sufficient to convert the gum base from individual pellets or balls into a semi-consistent, rubbery solid;
Adding CBD oil, mint oil, stevia, caffeine and/or gingerol, and fixing agent to the mixture;
Stirring the CBD oil, mint oil, caffeine and/or gingerol, stevia, fixing agent, and gum base together until a homogeneous rubbery solid develops;
Transferring said homogeneous rubbery solid to a conveyer belt;
Feeing said homogeneous rubbery solid through a compressing bottleneck in said conveyer belt, such that the resulting gum mixture is pressed into thin sheets;
Mechanically separating the thin gum sheets into small pieces fit for easy human chewing and use; and
Using a fixing agent to form a hard outer shell on the gum.

In the above embodiment, the gum base may be mixed for a duration ranging from 15-90 minutes with a temperature range of 75-180 C. The above embodiment is preferably made with ingredient proportions as laid out in Table 2, above.

The foregoing examples have been provided merely for the purpose of explanation and are in no way to be construed as limiting of the cannabis extract chewing gum, disclosed herein. While the cannabis extract chewing gum has been described with reference to various embodiments, it is understood that the words which have been used herein are words of description and illustration, rather than words of limitation. Further, although the cannabis extract chewing gum has been described herein with reference to particular means, materials, and embodiments, the cannabis extract chewing gum is not intended to be limited to the particulars disclosed herein; rather, the cannabis extract chewing gum extends to all functionally equivalent structures, methods and uses, such as are within the scope of the appended claims. Those skilled in the art, having the benefit of the teachings of this specification, may effect numerous modifications thereto and changes may be made without departing from the scope and spirit of the cannabis extract chewing gum disclosed herein in their aspects.

The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those of ordinary skill in the art that various modifications and variations may be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A cannabis extract chewing gum for delivering cannabinoids to a user, comprising:
a gum base;
one or more cannabis extracts;
one or more flavors;
one or more sweeteners; and
one or more additives.

2. The cannabis extract chewing gum of claim 1, wherein said one or more cannabis extracts is selected from the group of extracts consisting of tetrahydrocannabinol oil, cannabidiol oil, and combinations thereof.

3. The cannabis extract chewing gum of claim 1, wherein said one or more additives is selected from the group of extracts consisting of gingerol, caffeine, and combinations thereof.

4. The cannabis extract chewing gum of claim 1, wherein said one or more flavors is mint oil.

5. The cannabis extract chewing gum of claim 1, wherein said one or more sweeteners is stevia.

6. The cannabis extract chewing gum of claim 1, further comprising at least one fixing agent.

7. The cannabis extract chewing gum of claim 1, wherein said gum base is approximately 25.0-90.0 wt %, said one or more cannabis extracts is approximately 5.0-15.0% wt %, said flavoring is approximately 5.0-15.0 wt %, said sweetener is approximately 0.001-50.0 wt %, and said one or more additives is approximately 1.0-20.0 wt %.

8. The cannabis extract chewing gum of claim 7, wherein said gum base is approximately 65.0 wt %, said cannabis extract is approximately 10.0% wt %, said flavoring is approximately 10.0 wt %, said sweetener is approximately 5.0 wt %, and said additive is approximately 5-10.0 wt %.

9. A cannabis extract chewing gum for delivering cannabinoids to a user, comprising:
a gum base;
one or more cannabis extracts;
one or more flavors;
one or more sweeteners; and
one or more additives;
wherein said gum base is approximately 65.0 wt %;
wherein said one or more cannabis extracts is approximately 10.0% wt %;
wherein said one or more flavors is approximately 10.0 wt %;
wherein said one or more sweeteners is approximately 5.0 wt %; and
wherein said one or more additives is approximately 5.0-10.0 wt %.

10. The cannabis extract chewing gum of claim 9, wherein said one or more cannabis extracts is selected from the group of extracts consisting of tetrahydrocannabinol oil, cannabidiol oil, and combinations thereof.

11. The cannabis extract chewing gum of claim 10, wherein said one or more additives is selected from the group of additives consisting of gingerol, caffeine, and combinations thereof.

12. The cannabis extract chewing gum of claim 11, wherein said one or more sweeteners is stevia.

13. The cannabis extract chewing gum of claim 12, wherein said one or more flavors is mint oil.

14. The cannabis extract chewing gum of claim 13, further comprising at least one fixing agent.

15. A cannabis extract chewing gum for delivering cannabinoids to a user, comprising:
a gum base;
a cannabis extract;
mint oil;
one or more additives;
stevia; and
one or more fixing agents
wherein said gum base is approximately 65.0 wt %;
wherein said cannabis extract is approximately 10.0% wt %;
wherein said mint oil is approximately 10.0 wt %;
wherein said one or more additives is approximately 5.0-10 wt %; and
wherein said stevia is approximately 5.0 wt %.

16. The cannabis extract chewing gum of claim 15, wherein said cannabis extract is selected from the group of extracts consisting of tetrahydrocannabinol oil and cannabidiol oil.
